(19) United States
(12) Patent Application Publication
(10) Pub. No.: US 2022/0133965 A1
(43) Pub. Date: May 5, 2022

(54) INTERCONNECT DESIGN FOR JOINING DISSIMILAR MATERIALS

(71) Applicant: Medtronic, Inc., Minneapolis, MN (US)

(72) Inventors: Pankti N. Shah, Minneapolis, MN (US); John E. Hartung, Inver Grove Heights, MN (US); Hailiang Zhao, Plymouth, MN (US); Jason D. Hamack, Ramsey, MN (US); Amanda Travis, Maple Grove, MN (US)

(21) Appl. No.: 17/086,701

(22) Filed: Nov. 2, 2020

(51) Int. Cl. A61M 1/12

(52) U.S. Cl. CPC A61M 1/127; A61M 2205/8243; A61M 1/122

(57) ABSTRACT

A connector block for an implanted coil of a transcutaneous energy transfer system (TETS) includes a plurality of closed slots sized and configured to receive corresponding conductors of a powerline of the implanted coil. A plurality of open slots is included. The connector block being sized and configured to be coupled to the implanted coil.

### CROSS-REFERENCE TO RELATED APPLICATION

n/a.

### FIELD

The present technology is generally related to connector blocks for implanted transcutaneous energy transfer system (TETS) coils.

### BACKGROUND

Connector blocks are used to electrically connect conductors in an assembly. A connector block includes slots or openings to create a metal on metal contact with an exposed conductor. Conductors, however, are prone to fraying or warping out of the slots or openings in which they are supposed to be creating an electrical connection. Warping conductors can create numerous issues for an electronic device particularly those of a medical device. Warped or frayed conductors not fully in contact with a connector block or electric housing can cause a device to malfunction or short-circuit.

### SUMMARY

The techniques of this disclosure generally relate to connector blocks for implanted transcutaneous energy transfer system (TETS) coils.

In one aspect, a connector block for an implanted coil of a transcutaneous energy transfer system (TETS) includes a plurality of closed slots sized and configured to receive corresponding conductors of a powerline of the implanted coil. A plurality of open slots is included. The connector block being sized and configured to be coupled to the implanted coil.

In another aspect of this embodiment, the plurality open slots include a plurality of diameters.

In another aspect of this embodiment, each of the plurality of closed slots are spaced equidistant from an adjacent one of the plurality of closed slots.

In another aspect of this embodiment, each of the plurality of closed slots are configured for a tight fit with the corresponding conductors of the powerline.

In another aspect of this embodiment, the plurality of closed slots includes three closed slots each defining a same diameter.

In another aspect of this embodiment, the plurality of open slots includes a first open slot sized and configured to receive and retain at least a portion of the implanted coil.

In another aspect of this embodiment, the plurality of open slots includes a second open slot sized and configured to receive and retain at least a portion of a feedthrough pin.

In another aspect of this embodiment, at least one of the plurality of open slots defines an oblique-angled opening that facilitates a corresponding conductor to be press fit.

In another aspect of this embodiment, the oblique-angled opening is angled between 30°-60°.

In another aspect of this embodiment, a spacing between each of the plurality of open slots is larger than a spacing between each of the plurality of closed slots.

In one aspect, a transcutaneous energy transfer system (TETS) includes an implanted TETS coil. The implanted TETS coil includes a first connector block, the first connector block includes a plurality of closed slots sized and configured to receive corresponding conductors of a powerline and a plurality of open slots sized adjacent to the plurality of closed slots, the plurality of open slots are configured to receive at least one of a feed-through pin and a portion of the implanted TETS coil.

In another aspect of this embodiment, the system further includes a second connector block and a hermetic package coupled to the implanted TETS coil, the hermetic package being disposed between the first connector block and the second connector block.

In another aspect of this embodiment, the plurality open slots include a plurality of diameters.

In another aspect of this embodiment, each of the plurality of closed slots are spaced equidistant from an adjacent one of the plurality of closed slots.

In another aspect of this embodiment, each of the plurality of closed slots are configured for a tight fit with the corresponding conductors of the powerline.

In another aspect of this embodiment, the plurality of closed slots includes three closed slots each defining a same diameter.

In another aspect of this embodiment, at least one of the plurality of open slots defines an oblique-angled opening that facilitates a corresponding conductor to be press fit with the at least one open slot.

In another aspect of this embodiment, the oblique-angled opening is angled between 30°-60°.

In another aspect of this embodiment, a spacing between each of the plurality of open slots is larger than a spacing between each of the plurality of closed slots.

In one aspect, a connector block for an implanted coil of a transcutaneous energy transfer system (TETS) includes three closed slots sized and configured to receive conductors of a powerline of the implanted coil, each of the three closed slots defining a same diameter. Two open slots are aligned and adjacent with the three closed slots. A first of the two open slots is sized and configured to receive and retain a portion of the implanted coil, the first of the two open slots defines a first diameter. A second of the two open slots is sized and configured to receive a feedthrough pin, the second of the two open slots defines a second diameter small than the first diameter. The two open slots each define an oblique-angled opening of between 30-60 degrees.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS implanted coil constructed in accordance with the principles of the present application;

FIG. 2 is an external view of a TETS transmitter and a controller of the system shown in FIG. 1; and

FIG. 3 is a rear perspective view of the implanted coil shown in FIG. 1;

FIG. 4 is a front perspective view of the connector block shown in FIG. 4 showing various conductors engaged to the connector block; and

FIG. 5 a front view of the connector block show in FIG. 4.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

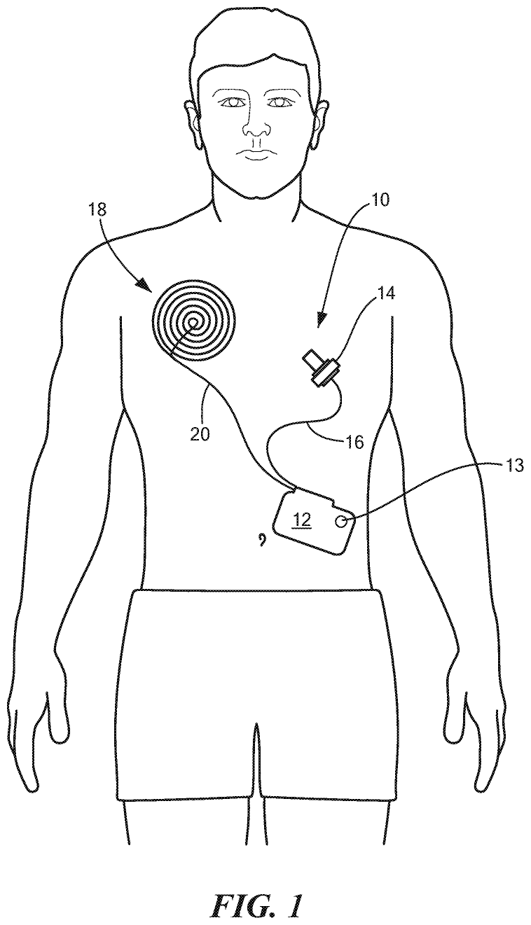

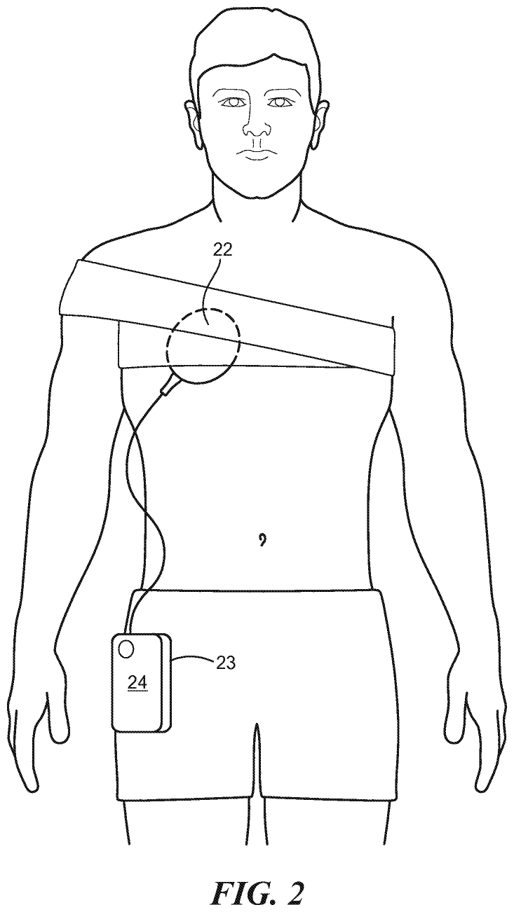

Referring now to the drawings in which like reference designators refer to like elements there is shown in FIGS. 1 and 2 an exemplary mechanical circulatory support device (“MCSD”) constructed in accordance with the principles of the present application and designated generally as “10.” The MCSD 10 may be fully implantable within a patient, whether human or animal, which is to say there are no percutaneous connections between the implanted components of the MCSD 10 and the components outside of the body of the patient. In the configuration shown in FIG. 1, the MCSD 10 includes an internal controller 12 implanted within the body of the patient. The internal controller 12 includes a control circuit having processing circuitry configured to control operation of an implantable blood pump 14. The internal controller 12 may include an internal power source 13, configured to power the components of the controller and provide power to one or more implantable medical devices, for example, the implantable blood pump, such as a ventricular assist device (“VAD”) 14 implanted within the left ventricle of the patient's heart. The power source 13 may include a variety of different types of power sources including an implantable battery. VADs 14 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609, the entirety of which is incorporated herein by reference. In an exemplary configuration, the VAD 14 is electrically coupled to the internal controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving or implanted coil 18 may also be coupled to the internal controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission or external coil 22 (seen in FIG. 2) disposed opposite the receiving coil 18 on the outside/exterior of the patient's body. For example, as shown in FIG. 2, a transmission coil 22 may be coupled to an external controller 23 having a power source 24, for example, a portable battery carried by the patient or wall power. In one configuration, the battery is configured to generate a radiofrequency signal for transmission of energy from the transmission coil 22 to the receiving coil 18. The receiving coil 18 may be configured for transcutaneous inductive communication with the transmission coil 22 to define a transcutaneous energy transfer system (TETS) that receives power from the transmission coil 22.

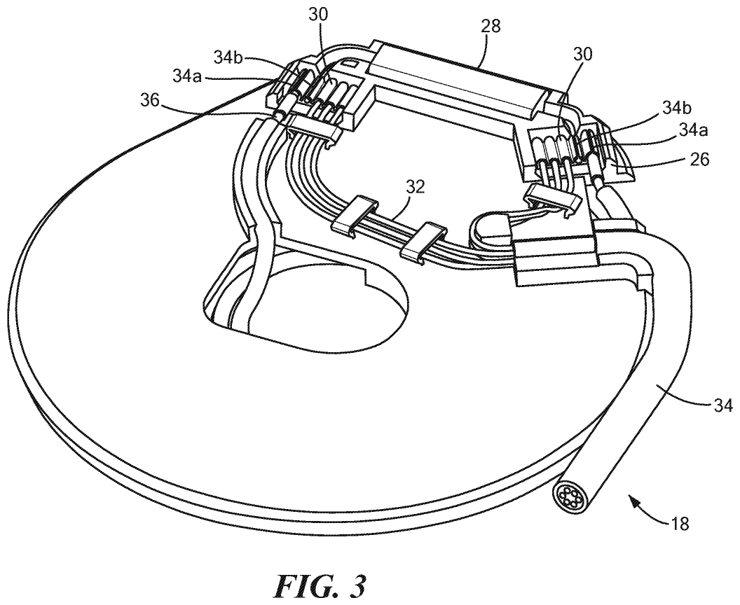

Referring now to FIG. 3 in which the implanted coil 18 is shown. The implanted coil 18 includes at least one connector block 26, sized and configured to be engaged to at least a portion of the implanted coil 18. In one configuration, the connector block 26 is recessed within a portion of the implanted coil 18. In some configurations, a second connector block 26 is included coupled to the implanted coil 18. In the configuration shown in FIG. 3, the connector blocks 26 are disposed on opposite sides of a hermetic package 28, which houses various electronics of the coil 18, including but not limited to capacitors, temperatures sensors, and one or more processors.

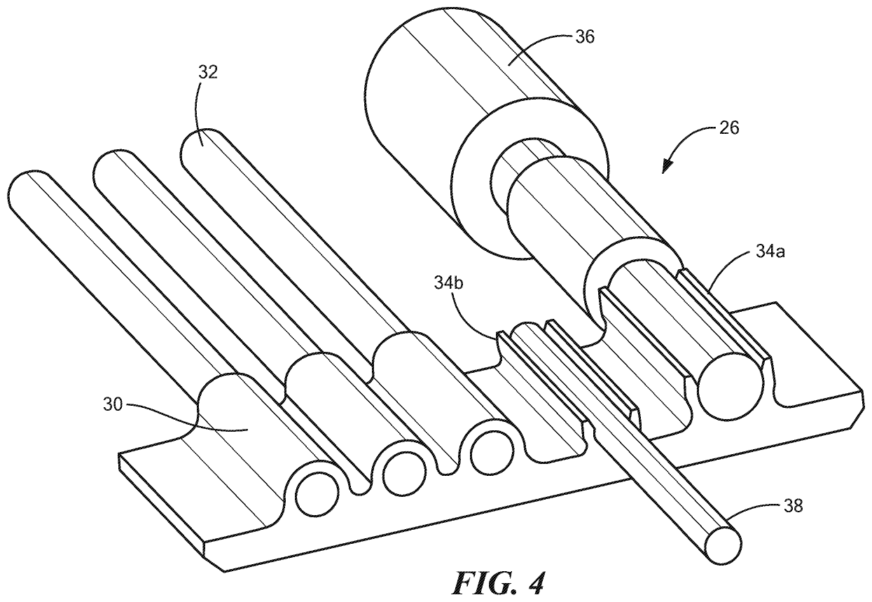

Referring now to FIGS. 3-5, the connector block 26 is sized and configured to receive and retain various conductors of the implanted coil 18. For example, the connector block, which may be composed of conductive materials, for example, titanium, niobium, or MP35N, is configured to mechanically and electrically connect the various electrical components of the implanted coil 18. To that end, the connector block 26 includes a plurality of closed slots 30 sized and configured to retain one or more conductors 32 of a powerline 34. In particular, the implanted coil 18 powers the VAD 14 by transferring power from the implanted coil 18 through the powerline 34 to the VAD 14. In an exemplary configuration, the power lines 34 includes three conductors 32, each conductor 32 being sized to be received and retained within a corresponding closed slot 30. In the configuration shown in FIG. 4, each conductor 32 is snug or tight fit within the corresponding closed slot 30. Each closed slot 30 may be equidistant from an adjacent closed slot 30 or may vary in distance as between adjacent closed slots 30 and each closed slot 30 may be the same diameter as an adjacent closed slot 30 or may vary in diameter.

Continuing to refer to FIG. 4, spaced a distance from the plurality of closed slots 30 are a plurality of open slots 34. In one configuration, two open slots 34 are included. A first of the open slots 34a defines a larger diameter than that of a second of the open slots 34b, although in other configurations the diameters of the open slots 34 are the same. The first open slot 34a is sized and configured to receive and retain a portion of the implanted coil 18. For example, the first open slot 34a is sized to receive a coil termination cap (CTC) 36 of the implanted coil 18. The CTC 36 may be snap-fit and welded within the first open-end slot 34a. The second open slot 34b is sized and configured to retain a feedthrough pin 38 which electrically connects the connector block 26 to the contents of the hermetic package 28. The feedthrough pin 38 may also be snap-fit within the second open slot 34b. In an exemplary configuration, the second open slot 34a defines a larger diameter than the of the second open slot 34b. Moreover, a spacing between the second open slot 34a and the second open slot 34b is greater than a spacing between adjacent ones of the plurality of closed slots 30.

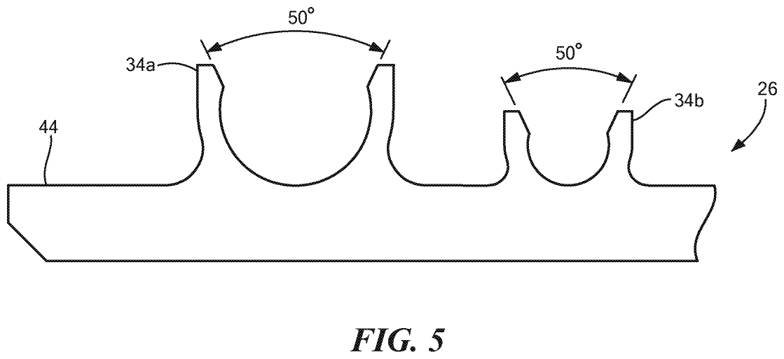

Referring now to FIG. 5, the plurality of open slots 34 each define an oblique-angled opening 40. The oblique-angled opening 40 defines an angle between 30 degrees and 60 degrees. That is, the plurality of open slots 34 each define a wall 42 that defines each slot 34. The wall 42 substantially defines an arcuate shape with the oblique-angled opening plus the circumference of the wall equaling 360 degrees. For example, in one configuration the wall defines a circumference of 310 degrees and the oblique-angled opening defines a circumference of 50 degrees to equal 360 degrees. To achieve an oblique-angled opening 40, each wall 42 is cut to define a wedge shape at the respective distal ends of the wall 42. This wedge shape enables the corresponding feedthrough pin 38 or CTC 36 to be snap fit and welded within the corresponding slot 34. Moreover, the connector block 26 may include a plurality of tabs 44 disposed on opposite sides of the connector block 26. The tabs 44 extend away from the connector block 26 which facilitate the connector block 26 being snapped into a portion of the implanted coil 18. In some configurations, the tabs 44 include a beveled edge which further facilitates engagement to a portion of the implanted coil 18.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A connector block for an implanted coil of a transcutaneous energy transfer system (TETS), comprising:
a plurality of closed slots sized and configured to receive corresponding conductors of a powerline of the implanted coil;
a plurality of open slots;
the connector block being sized and configured to be coupled to the implanted coil.

2. The connector block of claim 1, wherein the plurality open slots include a plurality of diameters.

3. The connector block of claim 1, wherein each of the plurality of closed slots are spaced equidistant from an adjacent one of the plurality of closed slots.

4. The connector block of claim 1, wherein each of the plurality of closed slots are configured for a tight fit with the corresponding conductors of the powerline.

5. The connector block of claim 1, wherein the plurality of closed slots includes three closed slots each defining a same diameter.

6. The connector block of claim 1, wherein the plurality of open slots includes a first open slot sized and configured to receive and retain at least a portion of the implanted coil.

7. The connector block of claim 1, wherein the plurality of open slots includes a second open slot sized and configured to receive and retain at least a portion of a feedthrough pin.

8. The connector block of claim 1, wherein at least one of the plurality of open slots defines an oblique-angled opening that facilitates a corresponding conductor to be press fit.

9. The connector block of claim 8, wherein the oblique-angled opening is angled between 30°-60°.

10. The connector block of claim 1, wherein a spacing between each of the plurality of open slots is larger than a spacing between each of the plurality of closed slots.

11. A transcutaneous energy transfer system (TETS), comprising:
an implanted TETS coil; and
the implanted TETS coil including a first connector block, the first connector block including:
a plurality of closed slots sized and configured to receive corresponding conductors of a powerline; and
a plurality of open slots sized adjacent to the plurality of closed slots, the plurality of open slots being configured to receive at least one of a feed-through pin and a portion of the implanted TETS coil.

12. The TETS of claim 11, further including a second connector block and a hermetic package coupled to the implanted TETS coil, the hermetic package being disposed between the first connector block and the second connector block.

13. The TETS of claim 11, wherein the plurality open slots include a plurality of diameters.

14. The TETS of claim 11, wherein each of the plurality of closed slots are spaced equidistant from an adjacent one of the plurality of closed slots.

15. The TETS of claim 11, wherein each of the plurality of closed slots are configured for a tight fit with the corresponding conductors of the powerline.

16. The TETS of claim 11, wherein the plurality of closed slots includes three closed slots each defining a same diameter.

17. The TETS of claim 11, wherein at least one of the plurality of open slots defines an oblique-angled opening that facilitates a corresponding conductor to be press fit with the at least one open slot.

18. The TETS of claim 17, wherein the oblique-angled opening is angled between 30°-60°.

19. The TETS of claim 11, wherein a spacing between each of the plurality of open slots is larger than a spacing between each of the plurality of closed slots.

20. A connector block for an implanted coil of a transcutaneous energy transfer system (TETS), comprising:
three closed slots sized and configured to receive conductors of a powerline of the implanted coil, each of the three closed slots defining a same diameter;
two open slots aligned and adjacent with the three closed slots;
a first of the two open slots being sized and configured to receive and retain a portion of the implanted coil, the first of the two open slots defining a first diameter;
a second of the two open slots being sized and configured to receive a feedthrough pin, the second of the two open slots defining a second diameter small than the first diameter; and
the two open slots each defining an oblique-angled opening of between 30-60 degrees.
